(19) United States
(12) Patent Application Publication
(10) Pub. No.: US 2023/0200787 A1
(43) Pub. Date: Jun. 29, 2023

(54) STOOL SAMPLE DEVICE

(71) Applicant: Region Sjælland, Sorø (DK)

(72) Inventors: Anders Ohlhues Baandrup, Frederiksberg C (DK); Anne-Dorte Krapper, Hedehusene (DK); Lasse Bremholm Hansen, Brøndby (DK)

(21) Appl. No.: 17/996,568

(22) PCT Filed: Apr. 19, 2021
(86) PCT No.: PCT/EP2021/060081

§ 371 Date: Oct. 19, 2022

(30) Foreign Application Priority Data
Apr. 21, 2020 (DK) PA202070244

(51) Int. Cl. A61B 10/00

(52) U.S. Cl. CPC A61B 10/0038; A61B 10/0096

(57) ABSTRACT

The invention relates to a stool sample device [A′] for collecting a stool sample from a region of a human rectum, characterised in that it comprises an outer part and an inner part, wherein the outer part comprises an outer unit [G″], wherein an inner unit [E″] is mounted, further wherein the outer unit [G″] has a round tip and comprises openings for collecting sample; and the inner part comprises the inner unit [E″] fitted with gaskets [D″], further wherein the inner unit [E″] is equipped with a spiral-shaped part or snail placed at the tip end relative to the outer unit [G″] and with a handle for turning [B″] and pulling [C″] at the opposite end relative to the spiral-shaped part or snail, a first gasket [D″] is placed at the tip end of the inner unit [E″], and a second gasket is placed at the opposite end of the inner unit [E″] relative to the spiral-shaped part or snail; wherein the inner unit [E″] can be turned and pulled back by the handle [B″, C″].

### FIELD OF THE INVENTION

The present invention relates to a stool sample device and a method for direct collection of an uncontaminated stool sample from the human rectum.

### BACKGROUND OF THE INVENTION

When a cancer patient in chemotherapy becomes hospitalised with acute diarrhea, it may be caused by several things, and in particular by 1) an infection with Clostridium Difficile (CD), 2) a chemotherapy-initiated diarrhea, or 3) other infectious diarrhea. This problem is large in all hospitals worldwide. CD is highly contagious, and in order to avoid spread of infection, it is therefore necessary to keep the patient in isolation, at least until the cause has been confirmed. While the patient is kept in isolation, a stool sample must be taken so that infection with either CD (or other types of contagious infectious induced diarrhea) or chemotherapy-initiated diarrhea can be confirmed, if one of these is the cause. This is the only way to have a confirmed diagnosis, and it is essential that the sample is not urine-contaminated or otherwise contaminated. As a result, the patients potentially end up being hospitalised up to three days due to stool samples that are urine-contaminated or otherwise contaminated, and the costs of hospitalisation for this reason are substantial. Accordingly, there is a need to find alternative ways of stool sampling and diagnosis in order to minimise the requirement for and the duration of hospitalisation due to diarrhea.

The above-mentioned scenario was the primary reason for the development of the present device, but the same field of problem applies for all isolated patients, where there is a need for uncontaminated stool samples, when they are hospitalised with a suspicion of potential, highly contagious gastrointestinal infections.

In addition, the device has a potential for use in other categories of hospitalised patients having suspected infections of contangious diarrhea, and who therefore need isolation; other potential area of use is in the primary care sector, where residents in eg. retirement facilities can have a quick and hygienic stool sample collected.

Chinese patent application CN2417832Y discloses a stool sample device for extracting a protected stool probe from a region of a human colon by insertion of the device into the colon, wherein the stool sampler comprises a cylindrical smooth body with a round head at the front end and a warning line, a toilet bowl, and a handle. After removal, the stool sample is protected from undesired contamination by being contained in the interior of the sample device until use and analysis. This method makes use of a scoop and prepares the sample for stool examination performed in the hospital.

The main object of the present invention is to provide a new stool sample device that has improved function in comparison with the prior art sampler because it is easier to handle, more gentle to the patient, and requires shorter diagnosis time.

Another object is to provide a sample device that can be manufactured in cheap materials, and is easy to manufacture, and that has a high degree of reliability.

It is also an object to provide an improved and fast method for diagnosis of diarrhoea and its cause.

### SUMMARY OF THE INVENTION

The present invention relates to a stool sample device [A″] for collecting a stool sample from a region of a human rectum, characterised in that it comprises an outer part and an inner part, wherein

the outer part comprises an outer unit [G″], wherein an inner unit [E″] is mounted, further wherein the outer unit [G″] has a round tip and comprises openings for collecting sample; and

the inner part comprises the inner unit [E″] fitted with gaskets [D″], further wherein the inner unit [E″] is equipped with a spiral-shaped part or snail placed at the tip end relative to the outer unit [G″] and with a handle for turning [B″] and pulling [C″] at the opposite end relative to the spiral-shaped part or snail, a first gasket [D″] is placed at the tip end of the inner unit [E″], and a second gasket is placed at the opposite end of the inner unit [E″] relative to the spiral-shaped part or snail; and

wherein the inner unit [E″] can be turned and pulled back by the handle [B″, C″].

In one embodiment of the invention, it relates to a stool sample device [A], wherein

the outer part comprises a first outer unit [G] and a second outer unit [H], each holding one of two separate inner units [E, F], and further wherein the two outer units [G, H] is either directly connected or is separated and connected by a cylindrical outer tube [I] surrounding an inner tube [J]; and

the inner part comprises the two separate inner units [E, F] each fitted with a gasket [D], wherein the first inner unit [E] is mounted within the first outer unit [G] and is equipped with the spiral-shaped part or snail placed at the tip end relative to the outer unit [G], and wherein the second inner unit [F] is mounted within the second outer unit [H] and is equipped with the handle for turning [B] and pulling [C], further wherein the two separate inner units [E, F] are connected by a cylindrical inner tube [J], the gasket [D] of the first inner unit [E] is placed at the tip end relative to the outer unit [G], and the gasket [D] of the second inner unit [F] is placed at the opposite end relative to the handle [B, C]; and

wherein the inner part consisting of the inner units [E, F] and the inner tube [J] can be turned and pulled back by the handle [B, C].

The present inventors have found that the invention can solve the problem of the requirement of prolonged hospitalisation. When a patient is admitted to the hospital with acute diarrhea expected to be infectious, and where there is a suspicion of CD or other contagious infectious induced cases of diarrhea, the hospital staff can collect a non-contaminated sample from a patient and analyse it substantially immediately to provide the sample test result within a very short time, such as within two or three hours. Accordingly, it does not take as much as three days to obtain a confirmation of the diagnosis as according to state of the art, and the hospital can easily, typically within three hours, find out if the patient will need to go into isolation or can be kept out of isolation.

The device of the invention can deliver a sample substantially immediately, and after sample analysis (two-three hours) the patient, if CD (or another diarrhea causing infection) was not the cause of diarrhea, can get out of hospital isolation. This quick analysis is more gentle to the patient than the method applied today, and at the same time it will save the hospital a lot of money.

The invention will also be applicable in other specialities and for screenings, and in a broader perspective, the device can be used in all clinical settings where patients in hospitals and residents in retirements facilities, within short notice, need to have stool samples taken that are not contaminated with urine or otherwise contaminated.

In addition, the device provides a significantly improvement in regards of hygiene and risk of contamination for the nursing staff who will be able to handle the potentially infectious stools in a much more confined area.

The present invention in another aspect relates to a method for collection of a uncontaminated stool sample from the human rectum, characterised in that the stool sample device according to any of the claims 1-10 is inserted into the human rectum, that the handle [B″, B, B′] of the device is turned a sufficient number of times to collect the sample, whereafter the handle [C″, C, C′] is pulled to a position wherein the inner unit [E″, E, E′] has passed the openings in the outer unit [G″, G, G′], and the gaskets [D″, D, D′] present in the inner units [E″, E, E′] and [F, F′] have sealed the device to hold the stool sample within the outer tube [I] or the outer unit [G″, H′], and the device is removed from the region of the human rectum.

Finally, the invention in a third aspect relates to a use of the stool sample device according to the invention for collecting a stool sample from a region of a human rectum.

### DRAWING

FIG. 1a illustrates the stool sample device and its components.

FIG. 1b illustrates the device three-dimensionally.

FIG. 1c illustrates an example of applicable dimensions of a stool sample device of the invention.

FIG. 2a illustrates a specific embodiment of the stool sample device of the invention.

FIG. 2b illustrates an example of applicable dimensions for this embodiment of the stool sample device of the invention.

FIG. 3 illustrates another specific embodiment of the stool sample device of the invention.

FIG. 4 shows the device in function in a hospital test with standard laboratory pigs.

### DETAILED DESCRIPTION OF THE INVENTION

In one embodiment of the invention in its three aspects, the inner unit(s) [E″, E, E′, F, F′] within the inner part of the stool sample device may be made up of a transparent material, such as a syntetic polymer, including polypropylene, e.g. nylon.

In a second embodiment of the invention in its three aspects, the gasket(s) [D″, D, D′] may be made of a silicium containing polymer, such as medical rubber silicone.

In any of the above embodiments of the invention, the outer unit(s) [G″, G, G′, H, H′] within the outer part may be made up of a transparent material, such as a syntetic polymer, including polypropylene, e.g. nylon.

In any of the above embodiments of the invention, the material of the outer unit (G″) or the outer tube [I, I′] may be flexible.

In any of the above embodiments of the invention, the material of the outer unit (G″) or the outer tube [I, I′] may be transparent.

In any of the above embodiments of the invention, the outer unit [G″, G, G′] may comprise 2, 3 or 4 openings for collecting sample, which openings may for example be square openings, such as elongated square openings, or round openings, such as circle- or oval-shaped openings. The openings are placed near to the tip end and assist to avoid excess pressure or vacuum during the stool sampling; in addition, the openings assist to avoid that the inner tube slides within the outer tube during the stool sampling.

In any of the above embodiments of the invention, the outer part may comprise a stop line for insertion of the device into the human rectum.

In one embodiment of the invention, a first outer unit [G′] and a second outer unit [H′] may be directly connected, and the second outer unit [H′], holding a second inner unit [F′], may have a cut thread in the opposite end relative to the handle [B′], and the first outer unit [G′] holding a first inner unit [E′] may hold a screw for attachment to the second outer unit [H′] in the cut thread. In this embodiment it is secured that the outer units [G′] and [H′] are hold firmly together when the device is removed from the human rectum.

In this embodiment of the invention, the second outer unit [G′] can be detached from the device at the attachment screw after extracting a stool probe and removing the device from the human rectum, thereby leaving the remaining part of the device holding the stool sample for sealing with a sealing [I′] at the opposite end relative to the handle [B′, C′].

In this embodiment, the remaining part of the device holding the stool sample may have means for being sealed with a shipments screw that can be mounted at the cut thread in the second outer unit [H′] left after detachment of the second outer unit [G′].

These embodiments will allow for an easy sealing and handling of the stool probe that is to be send to analysis and diagnosis of the cause of diarrhoea.

The skilled person will know how to choose the dimensions of the device so that they fit the purpose.

In the method of the invention for collecting a stool sample, the handle [B″, B, B′] is turned a sufficient number of times to collect the sample. Typically, the collection of a sample will require 4 to 10 turns, such as 4, 5, 6, 7, 8, 9, or 10 turns, e.g. 8 turns. Following this, the handle [C″, C, C′] is pulled to a position wherein the inner unit [E″, E, E′] has passed the openings in the outer unit [G″, G, G′] so that no more stool is sampled, and the gasket(s) [D″, D, D′] present in the inner unit(s) [E″, E, E′] and [F, F′] have sealed the device to hold the stool probe with the outer tube [I, I′] or the outer unit [G″, H, H′], and the device is removed from the region of the human rectum.

### EXAMPLES

### Example 1

One embodiment of the invention is now described as an example with reference to the drawing.

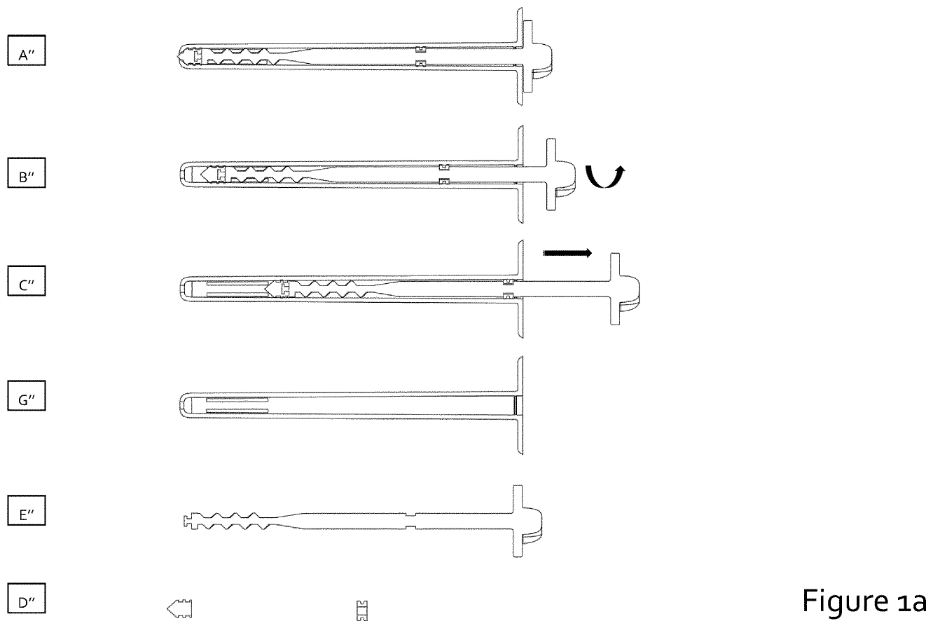

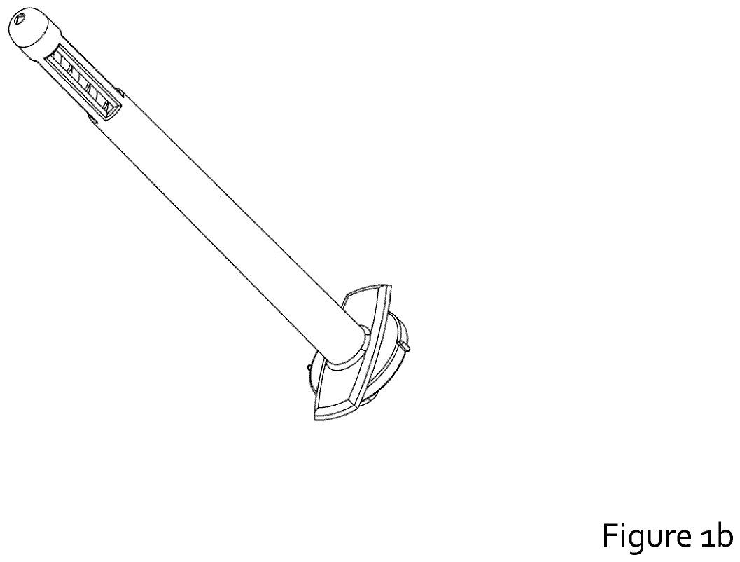

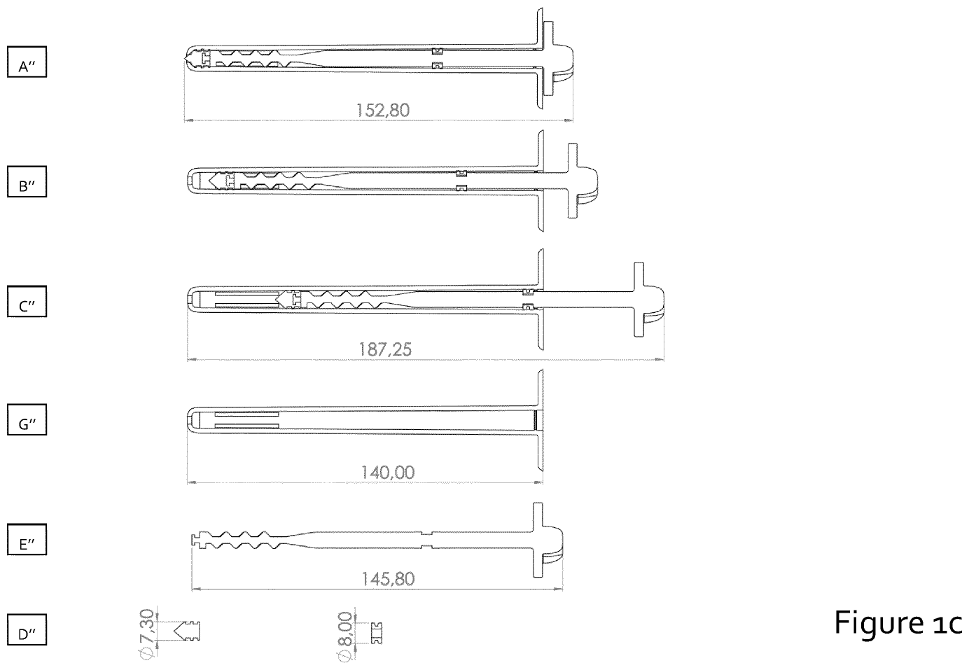

FIG. 1a shows the stool sample device [A″] as the ready-for-use device and the stool sample device when in use, [B″] and [C″], and the following details:

[B″] When the device [A″] has been inserted into the human rectum in the right position, the person in charge of sampling the stool sample rotates the handle [B″] a sufficient number of times to collect sufficient sample material, whereby the outer unit [G″] holding the inner unit [E″] is filled with sample material.

[C″] The handle can then be pulled to place the inner unit [E″] in a position passed the openings in the outer part [G″], and the sample material will be kept within the inner part [E″] which is sealed with the silicone gaskets [D″] present in [E″].

[E″] is the inner unit having a spiral-shaped part or snail that collects the sample material. The device is provided with silicone gaskets [D″] that seales the unit [E″] in the tip end and the opposite end of the device when the handle is pulled.

[G″] is the outer unit wherein the inner unit [E″] holding the spiral-shaped part or snail is mounted. Sample material enters through the unit's four elongated square openings.

### Example 2

FIG. 1c shows the same device and an example of applicable dimensions for the device.

In this embodiment of the device, the dimensions may be the following:

Length and diameter of the ready-for-use device: about 153 mm and about 8 mm;

Length of outer part G″: about 140 mm;

Length and diameter of inner part E″: about 146 mm.

### Example 3

Another embodiment of the invention is now described as an example with reference to the drawing.

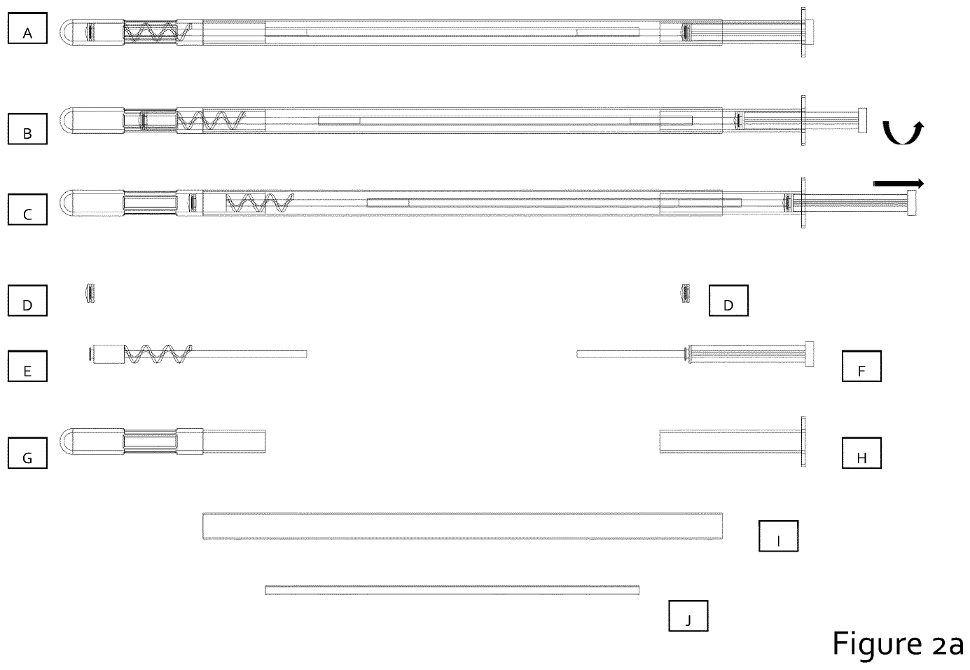

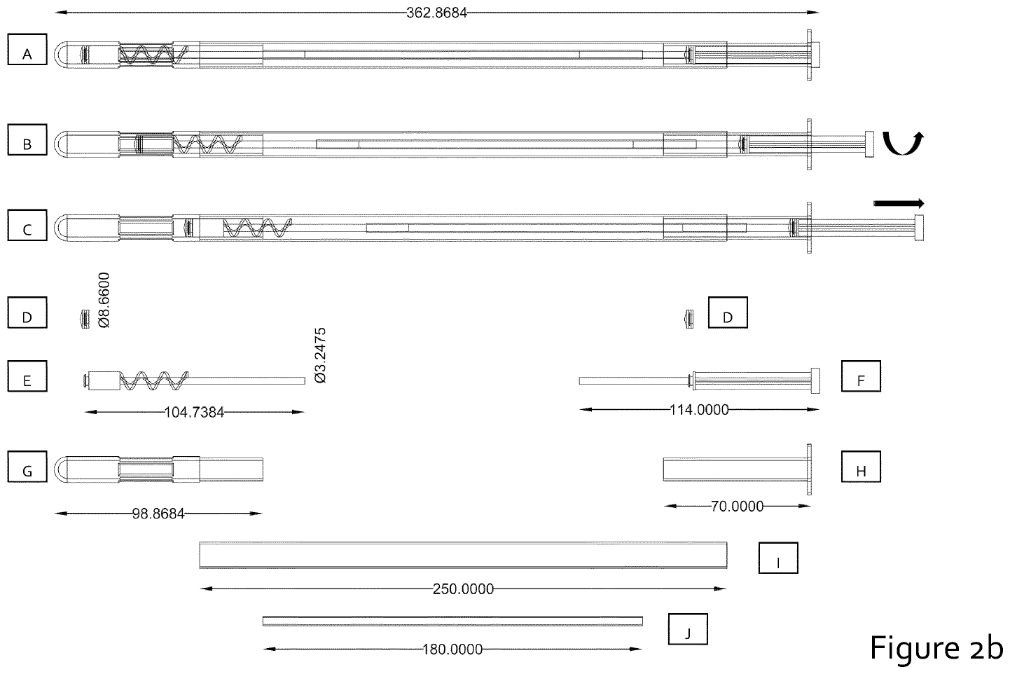

FIG. 2a shows the stool sample device [A] as the ready-for-use device and the stool sample device when in use, [B] and [C], and the following details:

[B] When the device [A] has been inserted into the human rectum in the right position, the person in charge of sampling the stool sample rotates the handle [B] a sufficient number of times to collect sufficient sample material, whereby the outer unit [G] holding the inner unit [E] and the outer tube [I] are filled with sample material.

[C] The handle can then be pulled to place the inner unit [E] in a position passed the openings in the outer part [G], and the sample material will be kept within the inner part [E] and the outer tube [I] which is sealed with the silicone gaskets [D] present in [E] and [F].

[E] is the inner unit having a spiral-shaped part or snail that collects the sample material. The device is provided with a silicone gasket [D] that seales the unit [E] in the tip end of the device when the handle is pulled.

[F] is the part of the device having a handle that can be turned and pulled by the user. The device is provided with a silicone gasket [D] that partly produces vacuum when the handle is pulled and which seals the end of the outer tube [I] opposite to the outer unit [G].

[G] is the outer unit wherein the inner unit [E] holding the spiral-shaped part or snail is mounted. Sample material enters through the unit's four elongated square openings.

[H] is the outer unit wherein the inner unit with the handle is mounted. [I] is the outer tube connecting the outer units [G] and [H]. [J] is the inner tube connecting the inner units [E] and [F].

### Example 4

FIG. 2b shows the same device and an example of applicable dimensions for the device.

In this embodiment of the device, the dimensions may be the following:

Length and diameter of the ready-for-use device: about 363 mm and about 12 mm;

Length of outer parts G, H and I: about 99 mm, about 70 mm, and about 250 mm, respectively;

Length and diameter of inner parts E, F, and J: about 105 mm, about 114 mm, and about 180 mm, respectively; diameter about 3.25 mm.

### Example 5

In one embodiment of the device the first outer unit [G′] holding the inner unit [E] can be 5 detached from the device after extracting a stool probe and removing the device from the human rectum.

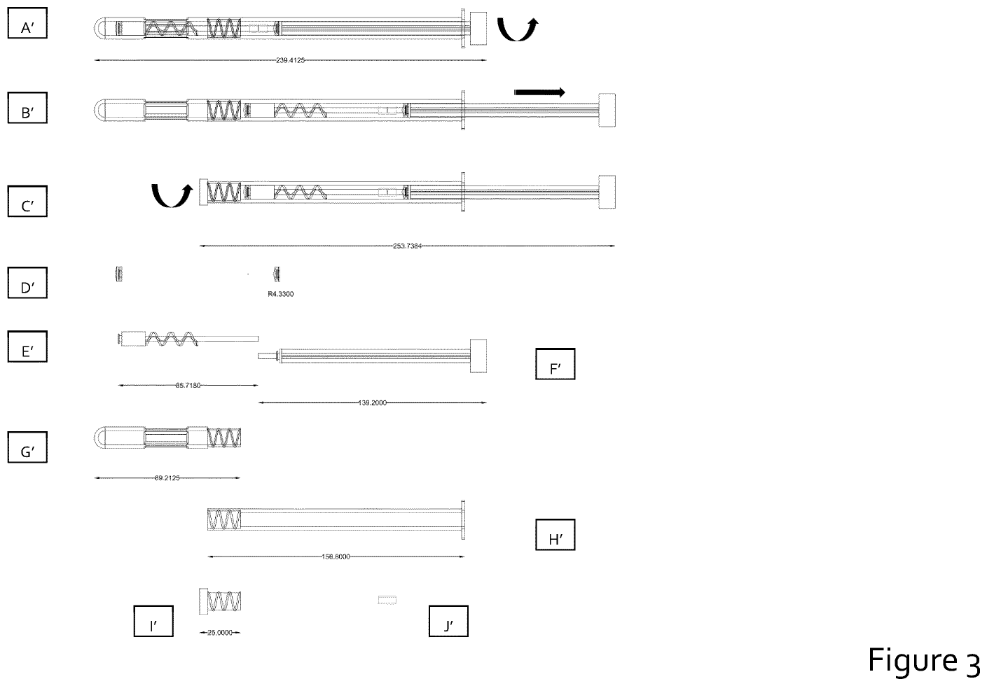

FIG. 3 shows the stool sample device [A′] as the ready-for-use device and the stool sample device when in use, [B′] and [C′], and the following details; in this embodiment the outer parts [G′] and [H′] is directly connected by a cut thread and a screw (there is no outer tube):

[A′] When the device has been inserted into the human rectum in the right position, the person in charge of sampling the stool sample rotates the handle a sufficient number of times to collect sufficient sample material, whereby the outer units [G′] and [H′] are filled with sample material.

[B′] The handle can then be pulled to place the inner unit [E′] in a position passed the openings in the outer part [G′], and the sample material will be kept within the inner part [E′] and the outer part [H′] which are sealed with the silicone gaskets [D′] present in [E′] and [F′].

[C′] The outer part [G′] can be removed and the end of the outer part [H′] is mounted with a shipments screw [I′].

[E′] is the inner unit having a spiral-shaped part or snail that collects the sample material. The device is provided with a silicone gasket [D′] that seales the unit [E′] in the tip end of the device when the handle is pulled.

[F′] is the part of the device having a handle that can be turned and pulled by the user. The device is provided with a silicone gasket [D′] that partly produces vacuum when the handle is pulled and which seals the end of the outer tube [H′] opposite to the outer unit [G′].

[G′] is the outer unit wherein the inner unit [E′] holding the spiral-shaped part is mounted. Sample material enters through the unit's four elongated square openings.

[H′] is the outer unit wherein the inner unit [F′] with the handle is mounted.

[J′] is the inner tube connecting the inner units [E′] and [F′].

### Example 6

A prototype of the device has in been tested on pigs in a controlled laboratory setting, under strict veterinarian control, and with the test animals in general anestesia. In this test the device proved functional, easily used, and performing in accordance to the theoretic expectations of the inventors.

### Test 1 of Rectal Stool Sample Device

### Conditions

Test site: University Hospital of Southern Denmark animal stables, operating theatre.

Test subjects: 2 standard laboratory pigs in general anesthesia, prepared and sedated for surgical training.

Test aim: Evaluation of prototype in relation to anatomy, user conditions, acquiring effect and safety in regards to mucosa damage and rectal lesions.

Description:

The test proto type was lubricated with standard rectal lubrication gel, the device was inserted in the rectum, and the “snail” was rotated clockwise, and retracted. Afterwards the rectum was inspected with a recto scope (devise for visual inspection). The procedure was documented by video and pictures. The procedure was completed with both test animals.

### Results

The device was easily and without resistance inserted, the “snail” was easily rotated. There was no faeces in the rectum, but the device obtained rectal mucus. The device was easily removed from the rectum. Subsequent inspection showed no harm to the rectal mucosa (the inside of the rectum), and there was no blood on the device indicating any rectal lesions.

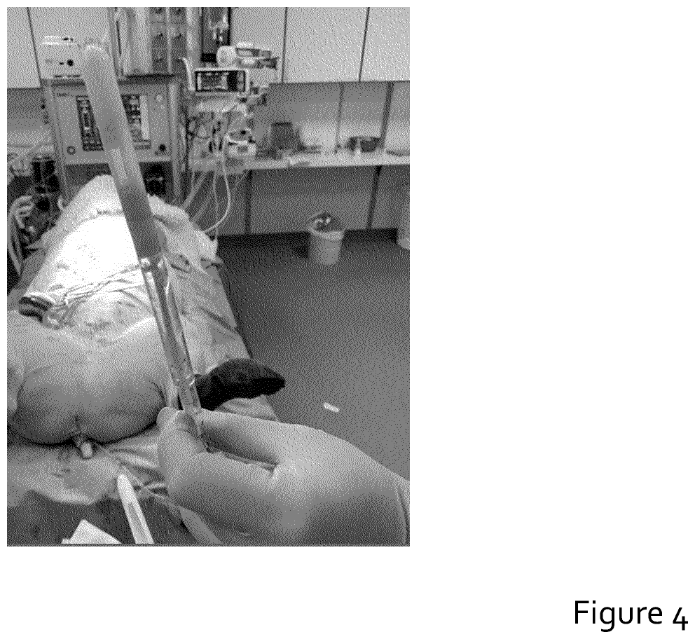

FIG. 4 shows the test device with mucus.

### Example 7

### Test 2 of Rectal Stool Sample Device

### Conditions

Test site: Zealand University Hospital

Test mannequin: Artificial human colon and rectum, in plastic and rubber, primarily designed to train in endoscopy.

Test aim: Evaluation of prototype in relation to acquiring effect.

Description:

The test prototype was lubricated with standard rectal lubrication gel, the device was inserted in the rubber rectum, and the “snail” was rotated clockwise, and retracted. A scope was inserted from the colon, and thereby was able to film the device in the artificial rectum. The rectum was filled with a liquid substance mimicking stool in the consistence of diarrhea. Video and pictures documented the procedure.

### Results

The procedure was repeated 10 times, and each time sufficient material was acquired.

1. A stool sample device for collecting a stool sample from a subject, wherein said stool sample device comprises an outer part and an inner part, wherein:
the outer part comprises an outer unit and an inner unit, wherein the inner unit is mounted to said outer unit and, wherein the outer unit comprises a round tip and openings for collecting sample; and
the inner part comprises the inner unit fitted with gaskets, wherein the inner unit comprises a spiral-shaped part or snail at the tip end relative to the outer unit and a handle configured for turning and pulling at the opposite end relative to the spiral-shaped part or snail, wherein a first gasket is placed at the tip end of the inner unit, and a second gasket is placed at the opposite end of the inner unit relative to the spiral-shaped part or snail; and
wherein the inner unit can be turned and pulled back by the handle.

2-13. (canceled)

14. The stool sample device according to claim 1, wherein:
the outer part comprises a first outer unit and a second outer unit, each holding one of two separate inner units, wherein the two outer units are either directly connected or are separated and connected by a cylindrical outer tube surrounding an inner tube; and
the inner part comprises the two separate inner units each fitted with a gasket, wherein the first inner unit is mounted within the first outer unit and is equipped with the spiral-shaped part or snail placed at the tip end relative to the outer unit, and wherein the second inner unit is mounted within the second outer unit and is equipped with the handle for turning and pulling, further wherein the two separate inner units are connected by a cylindrical inner tube, the gasket of the first inner unit is placed at the tip end relative to the outer unit, and the gasket of the second inner unit is placed at the opposite end relative to the handle; and
wherein the inner part consisting of the inner units and the inner tube can be turned and pulled back by the handle.

15. The stool sample device according to claim 1, wherein the inner unit(s) within the inner part, and/or the outer unit(s) within the outer part, comprise a transparent material.

16. The stool sample device according to claim 1, wherein the gasket(s) comprises a silicone containing polymer.

17. The stool sample device according to claim 1, wherein the material of the outer part or the outer tube is flexible.

18. The stool sample device according to claim 1, wherein the material of the outer part or the outer tube is transparent.

19. The stool sample device according to claim 1, wherein the outer unit comprises 2, 3, or 4 openings for collecting the sample, which openings are square openings, round openings , or oval-shaped openings.

20. The stool sample device according to claim 14, wherein the first outer unit and the second outer unit are directly connected, wherein the second outer unit, holding the second inner unit, has a cut thread in the opposite end relative to the handle, and wherein the first outer unit holding the first inner unit holds a screw for attachment to the second outer unit in the cut thread.

21. The stool sample device according to claim 14, wherein the first outer unit can be detached from the device at the attachment screw after extracting a stool probe and removing the device from the subject, thereby leaving the remaining part of the device holding the stool sample for sealing with a seal at the opposite end relative to the handle.

22. The stool sample device according to claim 21, wherein the remaining part of the device holding the stool sample is configured to be sealed with a shipments screw that is mounted at the cut thread in the second outer unit left after detachment of the first outer unit.

23. A method for collection of a uncontaminated stool sample from a subject, comprising:
inserting the stool sample device of claim 1 into the rectum of a subject,
turning the handle of the device to collect a stool sample, whereafter the handle is pulled to a position wherein the inner unit has passed the openings in the outer unit, and the gaskets present in the inner units have sealed the device so as to hold the stool sample within the outer tube or the outer unit, and
removing the device from the rectum of the subject.

24. The method according to claim 23, wherein the handle is turned 4 to 10 times.

25. The method of claim 23, wherein the stool sample device of claim 14 is inserted into the rectum of the subject.
